(19) United States
(12) Patent Application Publication
(10) Pub. No.: US 2015/0133741 A1
(43) Pub. Date: May 14, 2015

(54) INTUBATION METHODS AND SYSTEMS

(71) Applicant: Sukhjit Gill, Oakbrook, IL (US)

(72) Inventor: Sukhjit Gill, Oakbrook, IL (US)

(21) Appl. No.: 14/539,426

(22) Filed: Nov. 12, 2014

Related U.S. Application Data
(60) Provisional application No. 61/902,948, filed on Nov. 12, 2013.

(51) Int. Cl. A61M 16/04; A61B 1/06

(52) U.S. Cl. CPC A61M 16/0497; A61M 16/0463; A61B 1/0676; A61M 2205/8206
USPC 600/249; 128/200.26

(57) ABSTRACT

A system and a method for facilitating the entry of a tracheal airway of a patient during intubation are disclosed. The system of the present invention comprises an endobronchial tube and a support probe that is configured to be insertable within the endobronchial tube, wherein the support probe is configured to support the endobronchial tube. The length of the support probe is more than a length of the endobronchial tube to facilitate the entry of the endobronchial tube into the tracheal airway.

### CROSS-REFERENCES TO RELATED APPLICATIONS

This application claims the benefit and priority of U.S. Provisional Application No. 61/902,948, entitled “INTUBATION METHODS AND SYSTEMS”, filed on Nov. 12, 2013, the full disclosure of the above referenced application is incorporated herein by reference.

### BACKGROUND

The present disclosure relates generally to a medical device used in intubation of a patient, and more particularly to a system for facilitating the introduction of a tube into the patient's trachea.

Intubation is a method of securing the airway of a patient by inserting a flexible tube into the trachea of a patient through the mouth or nose, although it is done typically through the mouth. The most common conditions during which intubation may be required, include respiratory arrest/failure, airway obstruction due to multiple trauma, head injury, prolonged ventilator support, haemodynamic instability, abnormal mental status, pulmonary contusion, apnoea due to respiratory muscle weakness, and injury of vocal cords. Securing gaseous flow within the lungs of patients when they are in critical life-threatening diseases (like cardiovascular arrest) or when they are severely injured is vital, as failure to do so may result in subsequent failure of oxygenation and ventilation leading to brain damage, cardiac arrest and death.

Several types of intubation methods have been evolved, for example, endotrachial intubation, esophageal intubation, bronchial intubation, nasogastric intubation, nasotracheal intubation, tracheostomy intubation, orotracheal intubation, etc. In each intubation method, an intubating instrument in inserted into the trachea of a patient to prevent the obstruction of airway by providing free air flow into the lungs of a patient to ventilate the lungs. The intubating instrument, commonly referred as intubator, is mostly used when the patients do not maintain normal respiratory function on their own, particularly when they are unconscious. The intubating instrument can also be connected to mechanical ventilating machines for providing artificial respiration.

An intubation device with an endotracheal tube having thereon a scope with a light means and a tube clamp is disclosed in U.S. Pat. No. 5,163,941 to Garth et al. Similarly, U.S. Pat. No. 6,189,533 to Simon, et al., discloses an endotracheal intubation device having a light source at the distal end of the endotracheal tube.

Some of the problems caused during intubation are laceration of soft tissue, hematomas, edema, teeth abrasions, tongue swelling or macroglossia, tracheobronchial injuries and bronchial rupture leading to bronchial trauma. These may occur due to inappropriate tip design or shape, or contact of the tube tip with the trachea or nasal concha or turbinates. To avoid such problems instruments with flexible, tapered tip have been developed so as to enable smooth intubation. Further, the operation of some conventional camera/video assisted endotracheal intubation systems with in-built light source is challenging.

There still exists the need for a system that ensures safe and precise insertion of an endobronchial tube in an easy and secure manner.

### SUMMARY

Systems and methods for facilitating the entry of a tracheal airway are disclosed.

In one aspect, an intubating system for facilitating the entry of a tracheal airway is disclosed, wherein the system comprises an endobronchial tube and a support probe. The support probe is configured to be insertable into the endobronchial tube to facilitate the positioning of the endobronchial tube into the trachea of a patient. In one aspect, the length of the support probe is more than the length of the endobronchial tube. In another aspect, the support probe comprises a light source at the distal end, and is connected to a power source at the proximal end.

In yet another aspect, the support probe is of varying stiffness along the longitudinal body of the support probe, wherein the varying stiffness is achieved by at least one of a) varying a composition of the support probe, or b) varying a geometry of the support probe. In a different aspect, the support probe comprises a proximal opening at the proximal end and a distal opening at the distal end. The support tube further comprises a lumen disposed between the distal end and the proximal end. The proximal opening is configured to be connected to a vacuum source to create suction via the lumen. In one aspect, the proximal opening is configured to be connected to a fluid source configured to deliver a fluid via the lumen through the distal opening. In one aspect, the support probe comprises a material that is more rigid than the endobronchial tube.

In yet another aspect, a method of facilitating the entry of a tracheal airway is disclosed, wherein the method comprises placing an endobronchial tube into the mouth of a patient, and inserting a support probe into the endobronchial tube. The support probe is pushed against the endobronchial tube to advance the endobronchial tube into the tracheal airway. The support probe is removed after positioning the endobronchial tube into the trachea of the patient.

In one aspect, the method comprises illuminating the endobronchial tube using a light source disposed on the distal end of the support probe. The support tube further comprises a distal opening on the distal end and a proximal opening on the proximal end. In another aspect, the method comprises delivering suction through the support probe through the distal end of the support probe, and connecting a vacuum source to the proximal opening of the support probe. In a different embodiment, the method comprises delivering a fluid through the distal end of the support probe.

In still yet another aspect of the present embodiments relate to a support probe that is configured to aid the endobronchial tube to traverse the curvature in the airway smoothly, thereby forcing the epiglottis open.

In one aspect, the support probe is configured to enable precise control of endobronchial tube during the insertion process, facilitating easy manipulation of the endobronchial tube towards any direction.

The system and methods of the present invention allows a physician to simultaneously view the tracheal airway while positioning the endobronchial tube in place. The system of the present invention can be operable by even an inexperienced person with simple manipulation of the support probe.

Other aspects of the invention include corresponding methods, and systems are described herein.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A illustrates a perspective view of a device for facilitating the entry of tracheal airway of a patient in accordance with the present invention.

FIG. 1B illustrates one embodiment of the support probe of the present invention having a lumen disposed between the distal end and the proximal end of the support probe.

FIG. 1C-1F illustrate varying stiffness of the support probe of the present invention having a light source at the distal end of the support probe.

FIG. 2 illustrates the endobronchial tube of the present invention in its functioning position.

FIG. 3 is a flowchart describing a method for facilitating the entry of tracheal airway of a patient in accordance with one embodiment of the present invention.

FIG. 4 shows a method of inserting endobronchial tube into the trachea of a patient using the support probe in accordance with one embodiment of the present invention.

FIG. 5 is a flowchart describing a method for facilitating the entry of tracheal airway of a patient in accordance with another embodiment of the present invention.

FIG. 6 illustrates a method of inserting endobronchial tube into the trachea of a patient using the support probe beyond the length of the endobronchial tube in accordance with another embodiment of the present invention.

Referring to the drawings, like numbers indicate like parts throughout the views.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations

In one aspect, the present disclosure is directed to systems and methods for facilitating the entry of a tracheal airway. More specifically, in embodiment, present disclosure describes a system comprising an endobronchial tube and a support probe having a lumen therein, with other aspects including a light source, a power source, a fluid source and a vacuum source.

In another aspect, the present disclosure is directed to systems and methods that allow a physician to simultaneously insert the endobronchial tube and monitor the advancement of the endobronchial tube down the airway by controlling the movement of the support probe through the endobronchial tube.

In yet another aspect, the present disclosure is directed to systems and methods that provide a compact, portable assembly. In one embodiment, the system of the present disclosure is configured to prevent or minimize airway trauma associated with conventional endobronchial intubation procedures.

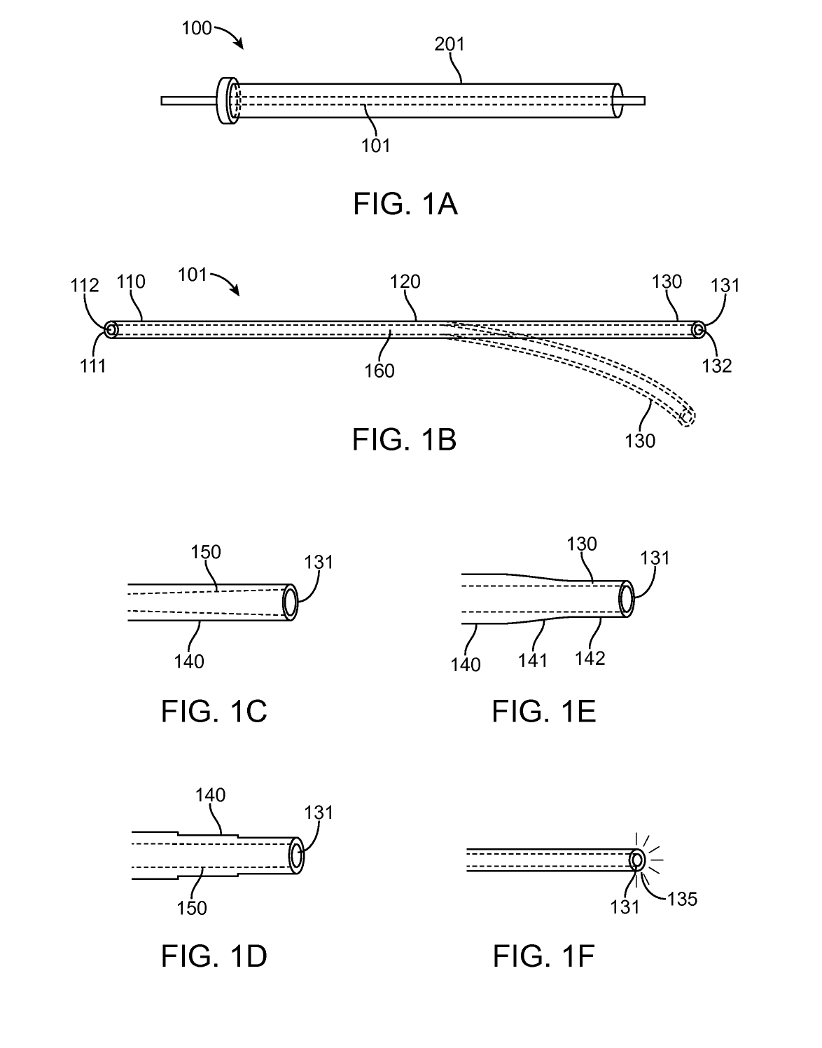

Aspects of the system 100 of the present disclosure is presented in various embodiments in FIGS. 1A to 1F. In one embodiment, the system 100 as shown in FIGS. 1A-1F comprises an endobronchial tube 201 and a support probe 101 that is configured to be insertable into the endobronchial tube 201 for guiding or aiding the movement of the endobronchial tube 201 through the airway of a patient. As shown in FIG. 1A, in some aspects, the length of the support probe 101 is longer than the length of the endobronchial tube 201 so as to enable proper positioning of the endobronchial tube 201.

In some aspects, the support probe 101 comprises a proximal part 110, a middle part 120 and a distal part 130. In one embodiment, the proximal part 110 has a proximal opening 112 at the proximal end 111 and the distal part 130 comprises a distal opening 132 at the distal end 131 as shown in FIG. 1B. In one embodiment, the distal part 130 of the support probe 101 is made flexible as shown in FIG. 1B so as to comfortably guide the endobronchial tube 201 over the curvature in the airway. In one aspect, the support probe 101 has a lumen 160 longitudinally disposed between the proximal end 111 and the distal end 131.

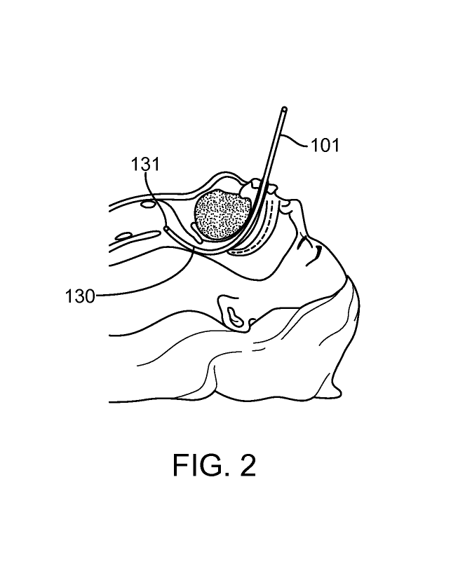

In one embodiment, the support probe 101 has variable stiffness along its length. For example, the support probe 101 may be stiffer at or near the proximal part 110, of intermediate stiffness at or near the middle part 120 and is relatively more flexible toward the distal part 130. In one embodiment, the distal end 131 is configured to be atraumatic to tissue. The variable stiffness in part 120 and part 130 are configured such that when the support probe is support and enable the probe as it traverses through the endobronchial tube, for example, the variable stiffness of the probe is configured to be capable of assuming a curvature such that the distal end 131 is placed within or points to the airway of a patient, as shown in FIG. 2.

The variable stiffness of support probe 101 illustrated in FIG. 1B and FIG. 2 is attained in various embodiments either by varying its composition or its geometry as shown in FIGS. 1C-1E. In one embodiment shown in FIG. 1C, the internal surface 150 of the probe 101 tapers along its length so as to provide a decreasing stiffness toward distal tip 131. In alternative embodiments (not shown), the reducing stiffness of probe 101 toward distal tip 131 is achieved by a taper along the external surface 140 or along both surfaces 140 and 150. In one embodiment shown in FIG. 1D, the external surface 140 of the probe 101 is shown with discontinuous or stepwise reducing diameter of exterior surface 140. In another embodiment shown in FIG. 1E, probe 101 is configured with decreasing stiffness with exterior surface 140 being provided with a tapered portion 141 extending therefrom, followed by a reduced diameter portion 142 near the distal tip 131. As illustrated in the examples in FIG. 1C to FIG. 1E, the probe 101 could be provided with variable stiffness towards the distal part 130 in several ways so as to achieve the desired shape when placed in the anatomy as shown in FIG. 2.

In some aspects the support probe 101 may be a solid probe without any lumen traversing the length of the probe.

In various embodiments, the materials detailed above could be used in suitable combinations to achieve variable stiffness along the length, as described earlier. For example, in some aspects, portion 110 of probe 101 is made of a rigid polymer, while portion 120 and particularly, portion 130 comprises greater proportions of a more flexible polymer or elastomer to provide the desired variable stiffness. Additionally or alternatively, in some embodiments the probe 101 is configured to incorporate a sleeve or lining of soft material such as an elastomeric polymer on the exterior surface 140, particularly at the distal portion 130 and the distal tip 131 in a manner as may be necessary to provide the necessary stiffness characteristics to the probe.

In some embodiments the support probe 101 comprises a distal portion 130 and distal tip 131 with soft atraumatic characteristics. In one embodiment, outer surface 140 of distal portion 130 with a coating of soft polymer that would be atraumatic when pushed against tissue. In some aspects, the support probe 101 comprises a light source 135 provided at the distal end 131 as shown in FIG. 1F to illuminate and to view a patient's body portion.

In some aspects, the light source 135 is configured to emit light as laterally directed path from the source. In one embodiment the light source 135 comprises one or more light emitting diode (LED) or organic light emitting diode (OLED). In another embodiment, the light source 135 comprises one or more of stacked organic light emitting diode (SOLED), flexible organic light emitting diode (FOLED), transparent organic light emitting diode (TOLED), and/or a phosphorescent organic light emitting diode (PHOLED).

In one embodiment, the light source 135 provided at the distal end 131 of the support probe 101 is configured to illuminate the airway to enable correct placement of the endobronchial tube 201. A power source such as a battery (not shown) is provided at the proximal end 111 of the support probe 101 and electrically connected to the light source 135 using suitable leads. The light source 135 is powered by the power source so as to provide sufficient illumination during intubation. The power source may preferably be a battery with suitable switching element.

In one embodiment, the system 100 of the present disclosure comprises a vacuum or pressurized fluid source (not shown in the figures) connected to proximal opening 112 of the probe 101 and configured to apply either suction or fluid pressure at the distal opening 132.

In one aspect, the support probe 101 can be constituted of a combination of suitable thermoplastic or elastomeric materials. Suitable materials include rigid biocompatible polymer such as nylon, polyethylene terephthalate (PET), polyamide, polyimide, polyethylene block amide copolymer, polypropylene, or polyether ether ketone (PEEK) polymers, a more flexible biocompatible polymer including various soft thermoplastic polyolefins (polyethylene and polypropylene), polyurethanes, polyesters and other suitable thermoplastic polymers. The rigid and flexible polymers as referred to above can be used in combination as required. In some embodiments elastomeric materials such as silicone, natural rubber, chloroprene rubber, and polyurethane can also be used as atraumatic sheath or coating in distal portion 130 of probe 101.

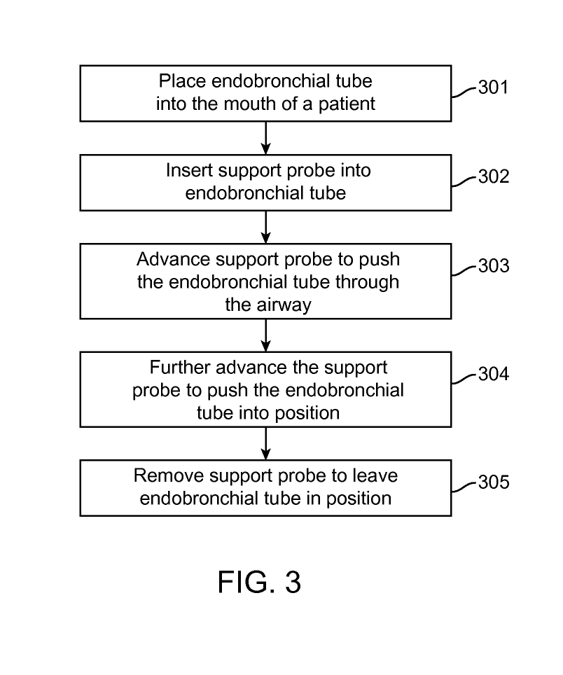

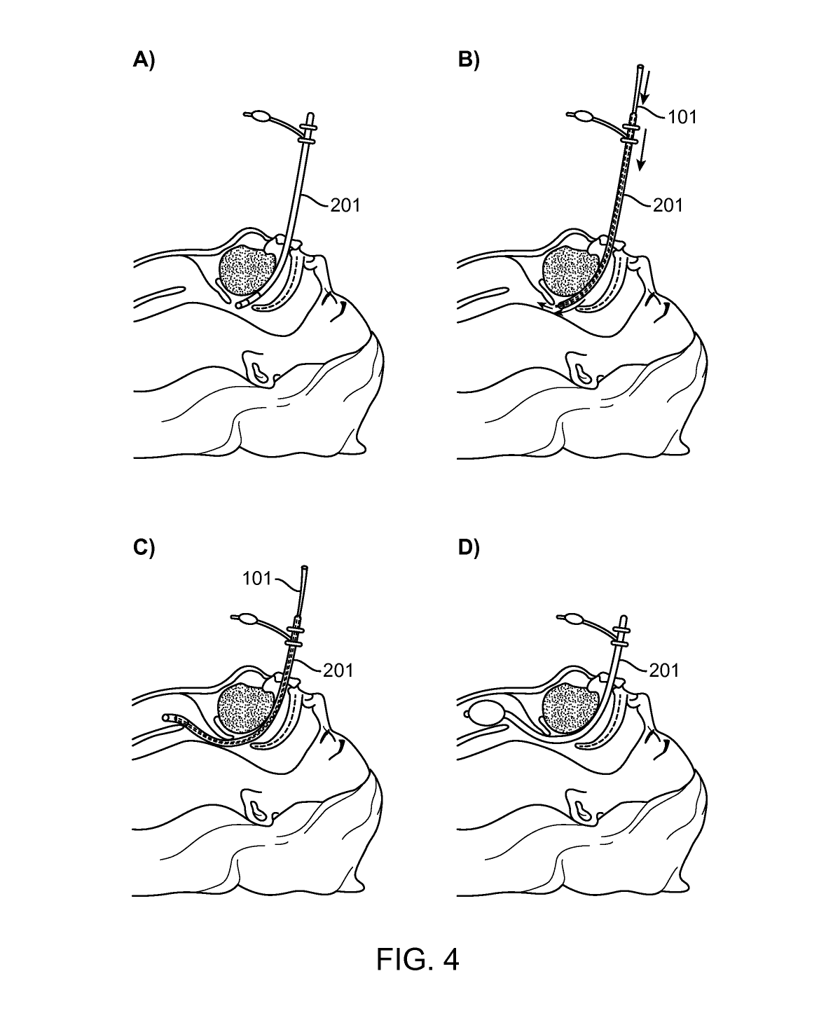

Referring now to FIG. 3, where a flow chart illustrating one exemplary method of facilitating the entry of an endobronchial tube into a tracheal airway using the system is shown. The steps of FIG. 3 are further illustrated pictorially in FIGS. 4A to 4D.

In step 301, the endobronchial tube 201 is placed in the mouth of a patient and pushed down through the mouth of a patient and at least partially placed in the trachea as shown in FIG. 4A. Once the endobronchial tube 201 reaches the curvature in the airway, it may be difficult to advance the tube further down the trachea due to the flexible nature of the endobronchial tube 201. At step 302, the support probe 101 is inserted into the endobronchial tube 201. As previously described, the support probe 101 is configured with the sufficient stiffness and/or curvature to support the endobronchial tube 201 and to aid the endobronchial tube 201 to traverse through the airway. At step 303, the support probe 101 is further advanced through the endobronchial tube 201. Due to the stiffness or rigidity of the support probe 101 in comparison with the endobronchial tube 101, when the distal portion of the support probe 101 is in contact with a portion of the inner wall of the endobronchial tube 201, the further advancement of the support probe 101 pushes against the endobronchial tube 201 and thus causing the endobronchial tube 201 to advance further into the airway. In one example, in the event that it is difficult for the endobronchial tube 201 to navigate through a difficult airway region due to the curvature of the anatomy, the effect created by the support tube 101 when the probe 101 is advanced through the endobronchial tube 201 can push the endobronchial tube 201 past the difficult airway region such that the endobronchial tube traverse through the difficult airway region.

At step 304, the endobronchial tube 201 is pushed along with the support probe 101 in combination to guide the endobronchial tube 201 past the epiglottis as illustrated in FIG. 4B. As earlier explained and with reference to FIGS. 1 and 2, at least a portion of the support probe 101 is more rigid than the endobronchial tube 201 and is of contoured stiffness so that it readily assumes the desired shape shown in FIG. 2, so as to simplify the insertion of the endobronchial tube 201. With the endobronchial tube positioned within the trachea, at step 305, the support probe 101 is removed, leaving the endobronchial tube 201 in the required position. In an alternative embodiment, the support probe 101 may only be used to aid the navigation of the endobronchial tube 201 through a particular difficult region, once the endobronchial tube 201 advances past the difficult region, the support probe 101 is the removed, and the endobronchial tube 201 is advanced in place to complete the intubation procedure without the support of the of the probe 101.

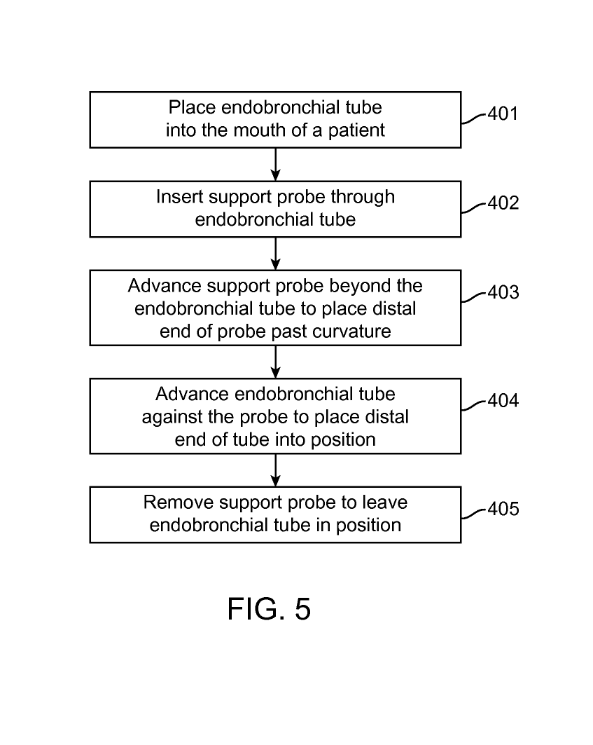

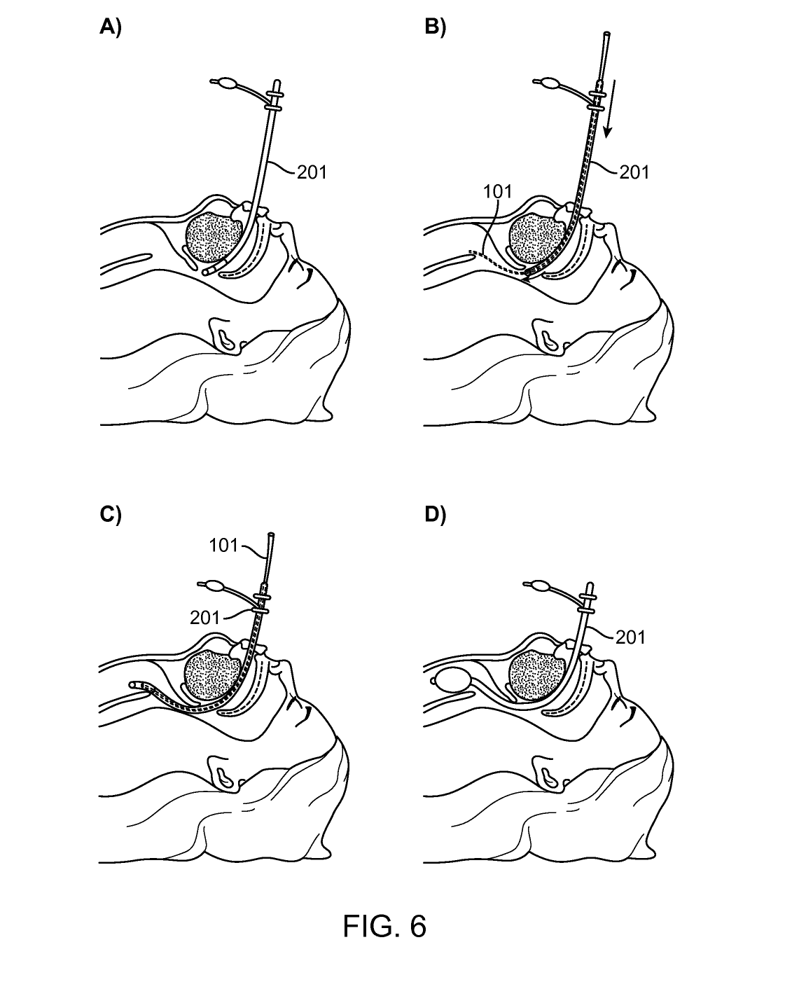

An alternative embodiment of the method for facilitating the entry of a tracheal airway using the system 100 of the present disclosure is shown as a flow chart in FIG. 5 and pictorially in FIGS. 6A to D. At step 401, the endobronchial tube 201 is first placed in the mouth of a patient as shown in FIG. 6A. At step 402, the support probe 101 is inserted through the endobronchial tube 201. At step 403, the support probe 101 is then advanced through the oropharynx, epiglottis, and vocal cords beyond the tip of the endobronchial tube 201 as shown in FIG. 6B. Once the support probe is placed pointing to the trachea, at step 404, the endobronchial tube 201 can be pushed over the probe 101 through the curvature in the airway into the trachea. In this position, distal end of endobronchial tube 201 is placed within the trachea of the patient. At step 405, the support probe is removed to leave the endobronchial tube in position.

Since in embodiment, the support probe 101 is longer than the longitudinal body of the endobronchial tube 201, the support probe 101 can be advanced beyond the endobronchial tube 201 to assist in the precise positioning of the same into the patient's trachea. The method of the present disclosure provides safe and facilitated positioning of the endobronchial tube 201 by handling the support probe 101 using simple and easy hand movement of the physician. After safe intubation, the support probe 101 of the present invention is withdrawn through the patient's mouth leaving the endobronchial tube 201 in position for continuation and completion of the medical procedure (step 405).

During the intubation procedure, the mucous and other accumulated secretions may have to be removed from the patient's airway as well as the lungs by applying vacuum or suction. The method of the present invention may additionally comprise creating suction via the lumen 160 using a suitably controlled vacuum source (not shown) coupled at the proximal opening 112 of the support probe 101. In some embodiments the method of the present disclosure may also comprise delivering a fluid via the lumen 160 and the distal opening 132 using a fluid source (not shown) coupled at the proximal opening 112. The fluid can be a suitable gas such as air or a liquid agent.

In some embodiments, a gripping device is disposed at the proximal end of the support probe 101 to enable firm gripping of the support probe 101 by the physician during the insertion through the endobronchial tube 201 and removal after proper positioning of the endobronchial tube 201. In another embodiment, a video camera may be attached near the light source 135 at the distal end 131 of the support probe 101. The video camera is configured to be in connection with a display screen such as a LCD screen provided at the proximal end 111 of the support probe 101. The video camera allows the physician to have a clear view of the airway through which the endobronchial tube 201 is advancing, thus facilitating exact placement of the endobronchial tube 201 in the proper location. The support probe 101 may optionally comprise a housing at the proximal end 111 to house the power source and other related features. The video camera can be a suitable solid state imaging device of appropriate resolution.

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

What is claimed is:

1. A system for facilitating the entry of a tracheal airway, comprising:
an endobronchial tube comprising a proximal end, a distal end, and a longitudinal body; and
a support probe comprising a proximal end, a distal end, and a longitudinal body disposed therebetween, wherein a length of the longitudinal body of the support probe is longer than a length of the longitudinal body of the endobronchial tube;
wherein the distal end of the support probe is configured to be insertable into the endobronchial tube and wherein the longitudinal body of the support probe is configured to support the endobronchial tube and facilitate the entry of the endobronchial tube into the tracheal airway.

2. The system of claim 1, wherein the support probe further comprises a light source disposed at its distal end.

3. The system of claim 2, wherein the support probe is configured to be connected to a power source.

4. The system of claim 1, wherein the support probe is of variable stiffness along its length and wherein the distal portion is more flexible than the proximal portion.

5. The system of claim 4, wherein the variable stiffness is provided by at least one of a) varying a composition of the support probe, or b) varying a geometry of the support probe.

6. The system of claim 1, wherein the support probe comprises a lumen disposed between the distal end and the proximal end and wherein the distal end comprises a distal opening and the proximal end comprises a proximal opening.

7. The system of claim 6, wherein the proximal opening is configured to be connected to a vacuum and configured to create suction via the lumen at the distal opening.

8. The system claim 6, wherein the proximal opening is configured to be connected to a fluid source configured to deliver fluid via the lumen and the distal opening.

9. The system of claim 1, wherein the support probe comprises a material that is more rigid than the endobronchial tube.

10. A method of facilitating the entry of a tracheal airway, comprising:
placing an endobronchial tube into the mouth of a patient;
inserting a support probe into the endobronchial tube, wherein the support probe comprises a proximal end, a distal end, and a longitudinal body disposed therebetween, wherein a length of a longitudinal body of the support probe is longer than a length of a longitudinal body of the endobronchial tube;
pushing the support tube against the endobronchial tube to advance the endobronchial tube into position in the tracheal airway; and
removing the support probe with the endobronchial tube in position in the tracheal airway.

11. The method of claim 10, wherein advancing the support probe comprises extending the support probe beyond the length of the endobronchial tube.

12. The method of claim 10, further comprising illuminating the endobronchial tube using a light source disposed on the distal end of the support probe.

13. The method of claim 10, further comprising delivering suction through the distal end of the support probe, wherein the support probe comprises a distal opening disposed on the distal end, a proximal opening disposed on the proximal end configured to be connected to a vacuum source and a lumen disposed therebetween.

14. The method of claim 10, further comprising delivering a fluid through the distal end of the support probe, wherein the support probe comprises a distal opening disposed on the distal end, a proximal opening disposed on the proximal end configured to be connected to a fluid source and a lumen disposed therebetween.

15. A method of facilitating the entry of a tracheal airway, comprising:
placing an endobronchial tube into the mouth of a patient;
inserting a support probe into the endobronchial tube, wherein the support probe comprises a proximal end, a distal end, and a longitudinal body disposed therebetween, wherein a length of a longitudinal body of the support probe is longer than a length of a longitudinal body of the endobronchial tube;
advancing the support probe through the endobronchial tube through the length of the tube whereby the distal end of the support probe is placed at the trachea;
advancing the endobronchial tube over the support probe into position in the tracheal airway; and
removing the support probe with the endobronchial tube in position in the tracheal airway.
